(19) United States
(12) Patent Application Publication
(10) Pub. No.: US 2019/0365321 A1
(43) Pub. Date: Dec. 5, 2019

(54) XR APPARATUS FOR PASSENGER IN VEHICLE

(71) Applicant: LG Electronics Inc., Seoul (KR)

(72) Inventor: Jinho SON, Seoul (KR)

(21) Appl. No.: 16/545,603

(22) Filed: Aug. 20, 2019

(30) Foreign Application Priority Data
Jun. 17, 2019 (KR) 10-2019-0071811

(51) Int. Cl. A61B 5/00; G02B 27/01; G02B 27/00; G06F 3/01

(52) U.S. Cl. CPC A61B 5/6803; G02B 27/017; G02B 2027/014; G06F 3/011; G02B 27/0093

(57) ABSTRACT

A method of providing XR contents for a passenger in a vehicle includes: checking whether or not the passenger has a specific disease based on the health information of the passenger on board the vehicle; receiving a condition monitoring result of the passenger from the vehicle; determining whether or not the passenger has an abnormal symptom based on the check result and the received condition monitoring result of the passenger; and displaying XR contents based on at least one of the determination result and the health information of the passenger.

### CROSS-REFERENCE TO RELATED APPLICATION

This application is based on and claims priority under 35 U.S.C. § 119(a) to Korean Patent Application No. 10-2019-0071811, filed on Jun. 17, 2019, the disclosure of which is incorporated herein in its entirety by reference.

### BACKGROUND

### 1. Field

The present disclosure relates to an XR apparatus for a passenger in a vehicle.

### 2. Description of the Related Art

A vehicle is transport means having only an internal combustion engine, and may include not only an automobile but also a train, a motorcycle, and the like. Traveling over long distances by the vehicle may cause fatigue to a passenger on board a vehicle. Particularly, in the case of a passenger suffering from a specific disease or severe motion sickness, the passenger may complain of an abnormal symptom while the vehicle is traveling. Accordingly, various contents that may be provided for the convenience of the passenger are under study.

Meanwhile, XR contents are contents that may provide a user with a new experience beyond the limitations of reality, and research thereof is being carried out for fusion and convergence of various fields.

### SUMMARY

The disclosed embodiments describes an XR apparatus and an XR contents providing method of providing eXtended Reality (XR) contents to a passenger, based on the health information and condition monitoring result of a passenger on board the vehicle. A technical problem to be dealt with by the present embodiment is not limited to the aforementioned technical problems, and other technical problems may be inferred from the following embodiments.

According to one aspect of the present invention, there is provided an eXtended reality (XR) apparatus for a passenger in a vehicle, including: a head mounted display (HMD) configured to monitor a condition of the passenger and to display XR contents based on the movement of the vehicle; a memory configured to store the XR contents; a processor configured to check whether or not the passenger has a specific disease based on the health information of the passenger, to determine whether or not the passenger has an abnormal symptom based on the check result and condition monitoring result of the passenger received from at least one of the HMD and the vehicle, and to make a request for XR contents based on at least one of the determination result and the health information of the passenger; and a communication unit connected to the processor and configured to transmit or receive a signal between the processor and at least one of an external network and the vehicle.

According to another aspect of the present invention, there is provided an XR apparatus for a passenger in a vehicle, including: a display including a plurality of display units attached to a vehicle, and configured to display extended reality (XR) contents based on a movement of the vehicle; a memory configured to store the XR contents; a processor configured to check whether or not the passenger has a specific disease based on the health information of the passenger, to determine whether or not the passenger has an abnormal symptom based on the checked result and condition monitoring result of the passenger received from the vehicle, to make a request for XR contents based on at least one of the determination result and the health information of the passenger, and to determine one or more display units included in the display as a display unit displaying the XR contents; and a communication unit connected to the processor, and configured to transmit or receive a signal between the processor and at least one of an external network and the vehicle.

According to still another aspect of the present invention, there is provided a method of providing XR contents for a passenger in a vehicle, including: checking whether or not the passenger has a specific disease based on health information of the passenger on board the vehicle; receiving a condition monitoring result of the passenger from the vehicle; determining whether or not the passenger has an abnormal symptom based on the received condition monitoring result of the passenger and the check result; and displaying XR contents based on at least one of the determination result and the health information of the passenger.

The specific matters of other embodiments are included in the detailed description and drawings.

According to an embodiment of the present invention, there is one or more of the following effects.

First, there is an effect that it is possible to alleviate an abnormal symptom caused by a specific disease or motion sickness of a passenger on board the vehicle.

Second, there is another effect that, in the case of an XR apparatus including a Head Mounted Display (HMD), it is possible to additionally monitor the condition of the passenger using not only a sensor included in the vehicle but also the HMD, thereby more accurately determining the abnormal symptom of the passenger.

Third, there is still another effect that, in the case of an XR apparatus including a plurality of displays attached to the vehicle, it is possible to alleviate an abnormal symptom caused by a specific disease or motion sickness even though a separate display device is not mounted on the passenger.

Fourth, there are still other effects that it is possible to control a travel speed of the vehicle based on the abnormal symptom of the passenger, or additionally control an air conditioning device, a directional spreading device, a lighting device, and a sound device of the vehicle, thereby further alleviating the abnormal symptom of the passenger.

The effects of the invention are not limited to the aforementioned effects, and other effects that have not been mentioned may be specifically understood by those skilled in the art from the description of the claims.

### BRIEF DESCRIPTION OF THE DRAWINGS

The above and other aspects, features, and advantages of certain embodiments will be more apparent from the following detailed description taken in conjunction with the accompanying drawings, in which:

FIG. 1 illustrates an AI device 100 according to an embodiment of the present disclosure.

FIG. 2 illustrates AI server 200 according to an embodiment of the present disclosure.

FIG. 3 illustrates an AI system 1 according to an embodiment of the present disclosure.

FIG. 4 is a view illustrating an example in which an HMD 420 of an XR apparatus according to an embodiment of the present invention provides a passenger with XR contents.

FIGS. 5A and 5B are views illustrating an example of an XR apparatus including a plurality of display units attached to a vehicle according to another embodiment of the present invention.

FIG. 6 is a block diagram of an XR apparatus 600 including an HMD according to an embodiment of the present invention.

FIG. 7 is a block diagram of an XR apparatus 700 including a plurality of display units attached to a vehicle according to another embodiment of the present invention.

FIG. 8 is a diagram illustrating a signal flow between an XR apparatus 800 for a passenger and external components 850 and 860 according to still another embodiment of the present invention.

FIG. 9 is a view illustrating an example in which an XR apparatus checks whether or not a passenger is suffering from a specific disease based on the health information of the passenger according to an embodiment of the present invention.

FIG. 10 is a view illustrating an example of a displayed message in a case where a predicted route of the vehicle includes a symptom-induced region according to an embodiment of the present invention.

FIG. 11 is a view illustrating an example of a displayed message in a case where a vehicle reaches a symptom-induced region according to an embodiment of the present invention.

FIG. 12 is a view illustrating an example of a displayed message in a case where it is determined that the abnormal symptom of the passenger appears according to an embodiment of the present invention.

FIG. 13 is a view illustrating an example in which relevant information is provided such that a passenger is able to take emergency measures in a case where the abnormal symptom of the passenger is not improved, according to an embodiment of the present invention.

FIG. 14 is a flowchart illustrating a method of providing XR contents for a passenger in a vehicle, according to an embodiment of the present invention.

FIG. 15 is a flowchart illustrating a method performed by the XR apparatus to avoid a symptom of a passenger in a case where a predicted travel route of the vehicle is delivered, according to an embodiment of the present invention.

FIG. 16 is a flowchart illustrating a method performed by the XR apparatus to alleviate an abnormal symptom of a passenger, according to another disclosed embodiment.

### DETAILED DESCRIPTION

In the following detailed description, reference is made to the accompanying drawing, which form a part hereof. The illustrative embodiments described in the detailed description, drawing, and claims are not meant to be limiting. Other embodiments may be utilized, and other changes may be made, without departing from the spirit or scope of the subject matter presented here.

Exemplary embodiments of the present invention are described in detail with reference to the accompanying drawings. Detailed descriptions of technical specifications well-known in the art and unrelated directly to the present invention may be omitted to avoid obscuring the subject matter of the present invention. This aims to omit unnecessary description so as to make clear the subject matter of the present invention. For the same reason, some elements are exaggerated, omitted, or simplified in the drawings and, in practice, the elements may have sizes and/or shapes different from those shown in the drawings. Throughout the drawings, the same or equivalent parts are indicated by the same reference numbers. Advantages and features of the present invention and methods of accomplishing the same may be understood more readily by reference to the following detailed description of exemplary embodiments and the accompanying drawings. The present invention may, however, be embodied in many different forms and should not be construed as being limited to the exemplary embodiments set forth herein. Rather, these exemplary embodiments are provided so that this disclosure will be thorough and complete and will fully convey the concept of the invention to those skilled in the art, and the present invention will only be defined by the appended claims. Like reference numerals refer to like elements throughout the specification. It will be understood that each block of the flowcharts and/or block diagrams, and combinations of blocks in the flowcharts and/or block diagrams, can be implemented by computer program instructions. These computer program instructions may be provided to a processor of a general-purpose computer, special purpose computer, or other programmable data processing apparatus, such that the instructions which are executed via the processor of the computer or other programmable data processing apparatus create means for implementing the functions/acts specified in the flowcharts and/or block diagrams. These computer program instructions may also be stored in a non-transitory computer-readable memory that can direct a computer or other programmable data processing apparatus to function in a particular manner, such that the instructions stored in the non-transitory computer-readable memory produce articles of manufacture embedding instruction means which implement the function/act specified in the flowcharts and/or block diagrams. The computer program instructions may also be loaded onto a computer or other programmable data processing apparatus to cause a series of operational steps to be performed on the computer or other programmable apparatus to produce a computer implemented process such that the instructions which are executed on the computer or other programmable apparatus provide steps for implementing the functions/acts specified in the flowcharts and/or block diagrams. Furthermore, the respective block diagrams may illustrate parts of modules, segments, or codes including at least one or more executable instructions for performing specific logic function(s). Moreover, it should be noted that the functions of the blocks may be performed in a different order in several modifications. For example, two successive blocks may be performed substantially at the same time, or may be performed in reverse order according to their functions. According to various embodiments of the present disclosure, the term “module”, means, but is not limited to, a software or hardware component, such as a Field Programmable Gate Array (FPGA) or Application Specific Integrated Circuit (ASIC), which performs certain tasks. A module may advantageously be configured to reside on the addressable storage medium and be configured to be executed on one or more processors. Thus, a module may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and modules may be combined into fewer components and modules or further separated into additional components and modules. In addition, the components and modules may be implemented such that they execute one or more CPUs in a device or a secure multimedia card. In addition, a controller mentioned in the embodiments may include at least one processor that is operated to control a corresponding apparatus.

In the present specification, a vehicle includes a vehicle having only an internal combustion engine, a hybrid vehicle having an internal combustion engine and an electric motor together, and an electric vehicle having only an electric motor, and includes not only an automobile but also a train.

At this time, the vehicle may include one or more sensor units capable of monitoring the condition of the passenger. The sensor unit of the vehicle may include a camera, light detection and ranging (LiDAR), a RADAR and a touch sensor, and may monitor passenger attitude, respiration, heart rate, body temperature, facial expression, It is not limited.

Artificial Intelligence refers to the field of studying artificial intelligence or a methodology capable of making the artificial intelligence. Machine learning refers to the field of studying methodologies that define and solve various problems handled in the field of artificial intelligence. Machine learning is also defined as an algorithm that enhances the performance of a task through a steady experience with respect to the task.

An artificial neural network (ANN) is a model used in machine learning, and may refer to a general model that is composed of artificial neurons (nodes) forming a network by synaptic connection and has problem solving ability. The artificial neural network may be defined by a connection pattern between neurons of different layers, a learning process of updating model parameters, and an activation function of generating an output value.

The artificial neural network may include an input layer and an output layer, and may selectively include one or more hidden layers. Each layer may include one or more neurons, and the artificial neural network may include a synapse that interconnects neurons. In the artificial neural network, each neuron may output input signals that are input through the synapse, weights, and the value of an activation function concerning deflection.

Model parameters refer to parameters determined by learning, and include weights for synaptic connection and deflection of neurons, for example. Then, hyper-parameters mean parameters to be set before learning in a machine learning algorithm, and include a learning rate, the number of repetitions, the size of a mini-batch, and an initialization function, for example.

It can be said that the purpose of learning of the artificial neural network is to determine a model parameter that minimizes a loss function. The loss function maybe used as an index for determining an optimal model parameter in a learning process of the artificial neural network.

Machine learning may be classified, according to a learning method, into supervised learning, unsupervised learning, and reinforcement learning.

The supervised learning refers to a learning method for an artificial neural network in the state in which a label for learning data is given. The label may refer to a correct answer (or a result value) to be deduced by an artificial neural network when learning data is input to the artificial neural network. The unsupervised learning may refer to a learning method for an artificial neural network in the state in which no label for learning data is given. The reinforcement learning may mean a learning method in which an agent defined in a certain environment learns to select a behavior or a behavior sequence that maximizes cumulative compensation in each state.

Machine learning realized by a deep neural network (DNN) including multiple hidden layers among artificial neural networks is also called deep learning, and deep learning is a part of machine learning. Hereinafter, machine learning is used as a meaning including deep learning.

In the present specification, an extended reality (XR) is collectively referred to as a virtual reality (VR), augmented reality (AR), and mixed reality (MR). VR technology provides real-world objects and backgrounds only as CG images, AR technology provides virtual images CG images on actual object images, and MR technology is a computer graphics technology that combines virtual objects in the real world.

MR technology is similar to AR technology in that it shows real and virtual objects together. However, in the AR technology, the virtual object is used as a complement to the real object, whereas in the MR technology, the virtual object and the real object are used in an equal manner.

XR technology can be applied to Head-Mounted Display (HMD), Head-Up Display (HUD), mobile phone, tablet PC, laptop, desktop, TV, digital signage. A device to which the XR technology is applied may be referred to as an XR apparatus.

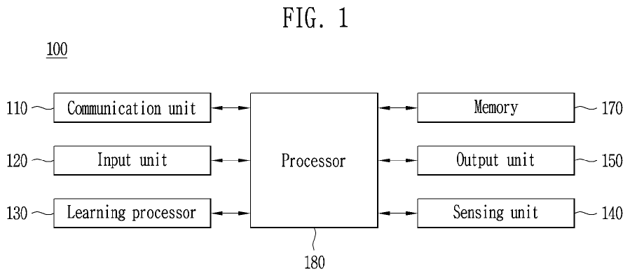

FIG. 1 illustrates an AI device 100 according to an embodiment of the present disclosure.

AI device 100 may be realized into, for example, a stationary appliance or a movable appliance, such as a TV, a projector, a cellular phone, a smart phone, a desktop computer, a laptop computer, a digital broadcasting terminal, a personal digital assistant (PDA), a portable multimedia player (PMP), a navigation system, a tablet PC, a wearable device, a set-top box (STB), a DMB receiver, a radio, a washing machine, a refrigerator, a digital signage, a robot, or a vehicle.

Referring to FIG. 1, Terminal 100 may include a communication unit 110, an input unit 120, a learning processor 130, a sensing unit 140, an output unit 150, a memory 170, and a processor 180, for example.

Communication unit 110 may transmit and receive data to and from external devices, such as other AI devices 100a to 100e and an AI server 200, using wired/wireless communication technologies. For example, communication unit 110 may transmit and receive sensor information, user input, learning models, and control signals, for example, to and from external devices.

At this time, the communication technology used by communication unit 110 may be, for example, a global system for mobile communication (GSM), code division multiple Access (CDMA), long term evolution (LTE), 5G, wireless LAN (WLAN), wireless-fidelity (Wi-Fi), Bluetooth™, radio frequency identification (RFID), infrared data association (IrDA), ZigBee, or near field communication (NFC).

Input unit 120 may acquire various types of data.

At this time, input unit 120 may include a camera for the input of an image signal, a microphone for receiving an audio signal, and a user input unit for receiving information input by a user, for example. Here, the camera or the microphone may be handled as a sensor, and a signal acquired from the camera or the microphone may be referred to as sensing data or sensor information.

Input unit 120 may acquire, for example, input data to be used when acquiring an output using learning data for model learning and a learning model. Input unit 120 may acquire unprocessed input data, and in this case, processor 180 or learning processor 130 may extract an input feature as pre-processing for the input data.

Learning processor 130 may cause a model configured with an artificial neural network to learn using the learning data. Here, the learned artificial neural network may be called a learning model. The learning model may be used to deduce a result value for newly input data other than the learning data, and the deduced value may be used as a determination base for performing any operation.

At this time, learning processor 130 may perform AI processing along with a learning processor 240 of AI server 200.

At this time, learning processor 130 may include a memory integrated or embodied in AI device 100. Alternatively, learning processor 130 may be realized using memory 170, an external memory directly coupled to AI device 100, or a memory held in an external device.

Sensing unit 140 may acquire at least one of internal information of AI device 100 and surrounding environmental information and user information of AI device 100 using various sensors.

At this time, the sensors included in sensing unit 140 may be a proximity sensor, an illuminance sensor, an acceleration sensor, a magnetic sensor, a gyro sensor, an inertial sensor, an RGB sensor, an IR sensor, a fingerprint recognition sensor, an ultrasonic sensor, an optical sensor, a microphone, a lidar, and a radar, for example.

Output unit 150 may generate, for example, a visual output, an auditory output, or a tactile output.

At this time, output unit 150 may include, for example, a display that outputs visual information, a speaker that outputs auditory information, and a haptic module that outputs tactile information.

Memory 170 may store data which assists various functions of AI device 100. For example, memory 170 may store input data acquired by input unit 120, learning data, learning models, and learning history, for example.

Processor 180 may determine at least one executable operation of AI device 100 based on information determined or generated using a data analysis algorithm or a machine learning algorithm. Then, processor 180 may control constituent elements of AI device 100 to perform the determined operation.

To this end, processor 180 may request, search, receive, or utilize data of learning processor 130 or memory 170, and may control the constituent elements of AI device 100 so as to execute a predictable operation or an operation that is deemed desirable among the at least one executable operation.

At this time, when connection of an external device is necessary to perform the determined operation, processor 180 may generate a control signal for controlling the external device and may transmit the generated control signal to the external device.

Processor 180 may acquire intention information with respect to user input and may determine a user request based on the acquired intention information.

At this time, processor 180 may acquire intention information corresponding to the user input using at least one of a speech to text (STT) engine for converting voice input into a character string and a natural language processing (NLP) engine for acquiring natural language intention information.

At this time, at least a part of the STT engine and/or the NLP engine may be configured with an artificial neural network learned according to a machine learning algorithm. Then, the STT engine and/or the NLP engine may have learned by learning processor 130, may have learned by learning processor 240 of AI server 200, or may have learned by distributed processing of processors 130 and 240.

Processor 180 may collect history information including, for example, the content of an operation of AI device 100 or feedback of the user with respect to an operation, and may store the collected information in memory 170 or learning processor 130, or may transmit the collected information to an external device such as AI server 200. The collected history information may be used to update a learning model.

Processor 180 may control at least some of the constituent elements of AI device 100 in order to drive an application program stored in memory 170. Moreover, processor 180 may combine and operate two or more of the constituent elements of AI device 100 for the driving of the application program.

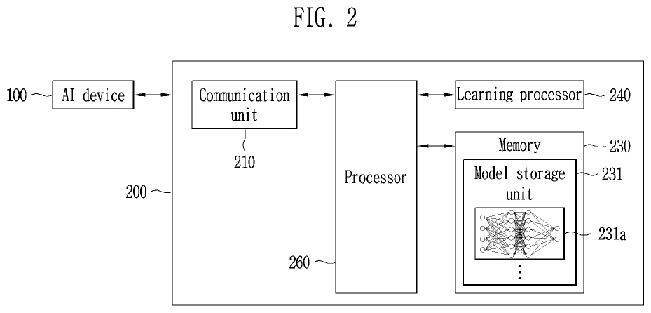

FIG. 2 illustrates AI server 200 according to an embodiment of the present disclosure.

Referring to FIG. 2, AI server 200 may refer to a device that causes an artificial neural network to learn using a machine learning algorithm or uses the learned artificial neural network. Here, AI server 200 may be constituted of multiple servers to perform distributed processing, and may be defined as a 5G network. At this time, AI server 200 may be included as a constituent element of AI device 100 so as to perform at least a part of AI processing together with AI device 100.

AI server 200 may include a communication unit 210, a memory 230, a learning processor 240, and a processor 260, for example.

Communication unit 210 may transmit and receive data to and from an external device such as AI device 100.

Memory 230 may include a model storage unit 231. Model storage unit 231 may store a model (or an artificial neural network) 231a which is learning or has learned via learning processor 240.

Learning processor 240 may cause artificial neural network 231a to learn learning data. A learning model may be used in the state of being mounted in AI server 200 of the artificial neural network, or may be used in the state of being mounted in an external device such as AI device 100.

The learning model may be realized in hardware, software, or a combination of hardware and software. In the case in which a part or the entirety of the learning model is realized in software, one or more instructions constituting the learning model may be stored in memory 230.

Processor 260 may deduce a result value for newly input data using the learning model, and may generate a response or a control instruction based on the deduced result value.

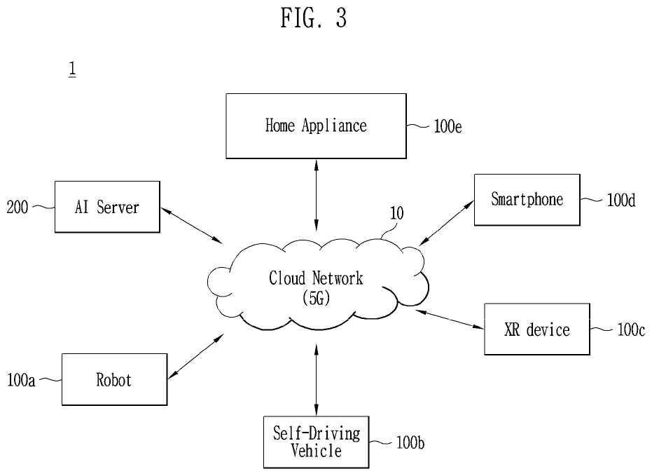

FIG. 3 illustrates an AI system 1 according to an embodiment of the present disclosure.

Referring to FIG. 3, in AI system 1, at least one of AI server 200, a robot 100a, an autonomous driving vehicle 100b, an XR apparatus 100c, a smart phone 100d, and a home appliance 100e is connected to a cloud network 10. Here, robot 100a, autonomous driving vehicle 100b, XR apparatus 100c, smart phone 100d, and home appliance 100e, to which AI technologies are applied, may be referred to as AI devices 100a to 100e.

Cloud network 10 may constitute a part of a cloud computing infra-structure, or may mean a network present in the cloud computing infra-structure. Here, cloud network 10 may be configured using a 3G network, a 4G or long term evolution (LTE) network, or a 5G network, for example.

That is, respective devices 100a to 100e and 200 constituting AI system 1 may be connected to each other via cloud network 10. In particular, respective devices 100a to 100e and 200 may communicate with each other via a base station, or may perform direct communication without the base station.

AI server 200 may include a server which performs AI processing and a server which performs an operation with respect to big data.

AI server 200 may be connected to at least one of robot 100a, autonomous driving vehicle 100b, XR apparatus 100c, smart phone 100d, and home appliance 100e, which are AI devices constituting AI system 1, via cloud network 10, and may assist at least a part of AI processing of connected AI devices 100a to 100e.

At this time, instead of AI devices 100a to 100e, AI server 200 may cause an artificial neural network to learn according to a machine learning algorithm, and may directly store a learning model or may transmit the learning model to AI devices 100a to 100e.

At this time, AI server 200 may receive input data from AI devices 100a to 100e, may deduce a result value for the received input data using the learning model, and may generate a response or a control instruction based on the deduced result value to transmit the response or the control instruction to AI devices 100a to 100e.

Alternatively, AI devices 100a to 100e may directly deduce a result value with respect to input data using the learning model, and may generate a response or a control instruction based on the deduced result value.

Hereinafter, various embodiments of AI devices 100a to 100e, to which the above-described technology is applied, will be described. Here, AI devices 100a to 100e illustrated in FIG. 3 may be specific embodiments of AI device 100 illustrated in FIG. 1.

The XR apparatus 100c according to the present embodiment can be applied to a head-mount display (HMD), a head-up display (HUD), a television, a mobile phone, a smart phone, a computer, a wearable device, Devices, digital signage, vehicles, fixed robots, mobile robots, and the like.

The XR apparatus 100c analyzes the three-dimensional point cloud data or image data acquired from various sensors or from an external device to generate position data and attribute data for the three-dimensional points, thereby obtaining information about the surrounding space or the real object and output the rendered XR object. For example, the XR apparatus 100c may output an XR object including the additional information about the recognized object, corresponding to the recognized object.

The XR apparatus 100c can perform the above-described operations using a learning model composed of at least one artificial neural network. For example, the XR apparatus 100c can recognize a real object from three-dimensional point cloud data or image data using a learning model, and can provide information corresponding to the recognized real object. Here, the learning model may be directly learned in the XR apparatus 100c, or learned in an external apparatus such as the AI server 200.

At this time, the XR apparatus 100c may generate the result using the direct learning model and perform an operation. However, the XR apparatus 100c may transmit the sensor information to an external device such as the AI server 200, and may receive the generated result and perform an operation.

In addition, the XR apparatus according to another embodiment may be a component of an autonomous vehicle. In other words, the autonomous vehicle 100b can be implemented as a mobile robot, a vehicle, an unmanned aerial vehicle, or the like.

The autonomous vehicle 100b to which the XR technique is applied may mean an autonomous vehicle having means for providing an XR image or an autonomous vehicle to be controlled/interacted in an XR image. Particularly, the autonomous vehicle 100b to be controlled/interacted within the XR image can be distinguished from the XR apparatus 100c and interlocked with each other.

The autonomous vehicle 100b having the means for providing the XR image can acquire the sensor information from the sensors including the camera and output the generated XR image based on the acquired sensor information. For example, the autonomous vehicle 100b may include an HUD to output an XR image, thereby providing an passenger with a real object or an XR object corresponding to an object in the screen.

At this time, when the XR object is outputted to the HUD, at least a part of the XR object may be outputted so as to overlap with the real object that the passenger's gaze is directed to. On the other hand, when the XR object is output to the display provided in the autonomous vehicle 100b, at least a part of the XR object may be output so as to overlap the object in the screen. For example, the autonomous vehicle 100b may output XR objects corresponding to objects such as a lane, another vehicle, a traffic light, a traffic sign, a motorcycle, a pedestrian, a building, and the like.

When the autonomous vehicle 100b to be controlled/interacted within the XR image acquires the sensor information from the sensors including the camera, the autonomous vehicle 100b or the XR apparatus 100c generates XR image based on the sensor information, and the XR apparatus 100c can output the generated XR image. The autonomous vehicle 100b may operate based on a control signal or an interaction of a user input through an external device such as the XR apparatus 100c.

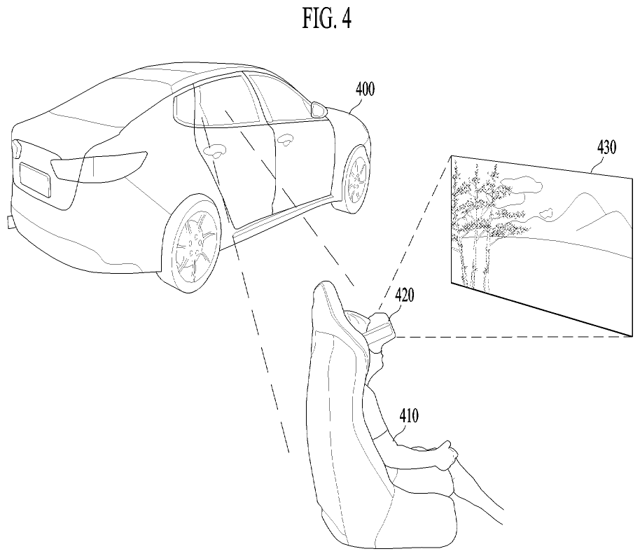

FIG. 4 is a view illustrating an example in which an HMD 420 of an XR apparatus provides a passenger with XR contents according to an embodiment of the present invention.

The XR apparatus according to an embodiment of the present invention may provide XR contents 430 to a passenger 410 on board the vehicle 400, by using the HMD 420. For example, in the case where the passenger 410 on the vehicle 400 is suffering from a specific disease, has a severe motion sickness, is a dementia patient, or is a child, the passenger 410 may have difficulty in traveling on board the vehicle 400 for a long time. In this case, the XR apparatus may receive the health information of the passenger 410 and condition monitoring result of the passenger 410 to determine whether or not the passenger 410 has an abnormal symptom while the vehicle 400 is traveling. Here, the XR apparatus may determine whether or not the passenger is suffering from a specific disease based on the health information of the passenger 410 received from an external network or the health information of the passenger 410 previously stored.

In addition, the XR apparatus may receive the condition monitoring result of the passenger 410 from at least one of the HMD 420 and the sensor unit of the vehicle 400. In the case where it is determined based on the delivered condition monitoring result of the passenger 410 that the passenger 410 has an abnormal symptom, the XR apparatus may provide XR contents 420 causing the abnormal symptom of the passenger 410 to be alleviated, through the HMD 420. At this time, the HMD 420 may reproduce XR contents based on the movement of the vehicle.

For example, according to the condition monitoring result of the passenger 410, when it is determined that the passenger 410 has an abnormal symptom such as breaking out in a cold sweat or bowing forward caused by motion sickness, the XR apparatus recommends the passenger 410 to wear the HMD 420, and may provide the passenger 410 with XR contents 430 including a broad plain image so that the motion sickness of the passenger may be alleviated.

When it is determined through the health information of the passenger 410 that the passenger is suffering from the claustrophobia, the XR apparatus may recommend the passenger 410 to wear the HMD 420, in the case where the passenger 410 has an abnormal symptom such as closing his/her eyes, bending his/her head back, and looking pale in his/her face while the vehicle 400 is driven. In addition, the XR apparatus may provide XR contents 430 including a bright outdoor image such that the passenger 410 has improvement in the abnormal symptom caused by the claustrophobia.

Meanwhile, in the case where the vehicle reaches the symptom-induced region, the XR apparatus may provide XR contents based on the health information of the passenger. Here, the symptom-induced region may be defined as an area in which the passenger is likely to have an abnormal symptom among areas included in a travel route of the vehicle. For example, in a case where it is determined through the health information of the passenger that the passenger is suffering from agoraphobia or claustrophobia, the symptom-induced region may be a night highway, a tunnel, a road with congested traffic, and the like in which agoraphobia or claustrophobia is likely to appear. In addition, in the case where it is determined through the health information of the passenger that the passenger has severe motion sickness, the symptom-induced region may be a curved section. However, a type of the symptom-induced region is not limited thereto. In addition, according to the embodiment, the symptom-induced region may be determined based on whether or not the symptom has been induced while another user has been traveling in the region.

In addition, the health information of the passenger on board in the embodiment may be received through an external server, an operation regarding condition monitoring may be performed in a manner that has been not described in the specification, and the XR apparatus may provide XR contents based on the received information.

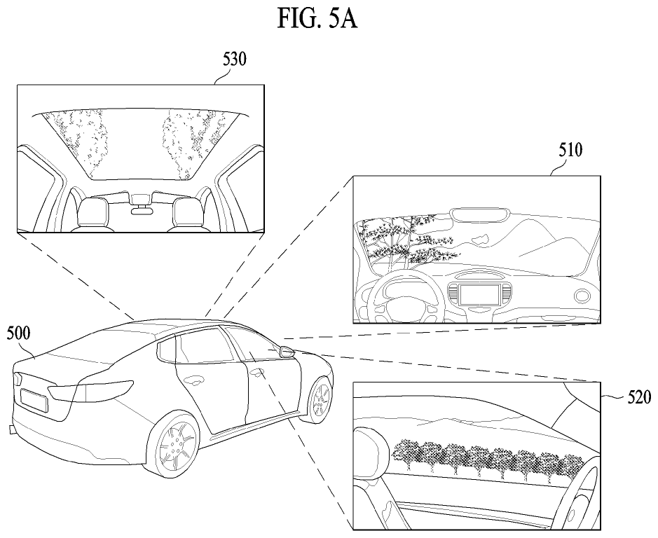

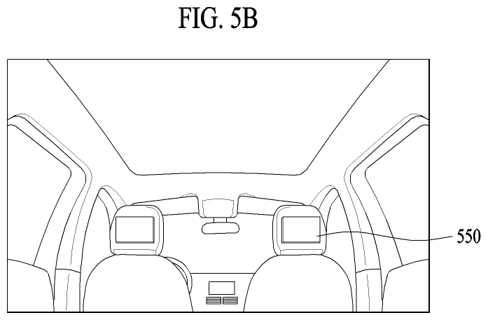

FIGS. 5A and 5B are views illustrating an example of an XR apparatus including a plurality of display units attached to a vehicle according to another embodiment of the present invention.

The XR apparatus according to an embodiment of the present invention may provide XR contents to passengers on board a vehicle 500, by using a plurality of display units attached to a windshield and a window of the vehicle 500, and a component inside the vehicle 500. Here, the plurality of display units may be collectively referred to as a display in the following description.

FIG. 5A is a view illustrating an example of display units 510, 520, and 530 attached to a windshield, both side windows and a ceiling of the vehicle 500, and FIG. 5B is a view illustrating an example of a display unit 550 attached inside the vehicle 500. The XR apparatus according to an embodiment of the present invention may select a display unit in which XR contents are displayed according to a position of the passenger. For example, in the case of providing XR contents to a passenger in a passenger seat, the XR apparatus may provide XR contents to the passenger through the display units 510, 520, and 530 attached to the windshield, both side windows, and ceiling of the vehicle 500 in FIG. 5A. In addition, in a case of providing XR contents to a passenger in a rear seat behind the passenger seat, the XR apparatus may provide XR contents to the passenger through the display units 520 and 530 attached to both side windows and the ceiling of the vehicle 500 in FIG. 5A and through a display unit 550 on the rear side of the passenger seat inside the vehicle in FIG. 5B.

In addition, in the embodiment, it is possible to determine a display on which XR contents are provided based on whether or not the vehicle is in an autonomous driving mode. More specifically, in a case where the vehicle is not in the autonomous driving mode, XR contents may be reproduced on displays other than a display related to an element for obtaining information for traveling. In addition, in the case where the vehicle is in the autonomous driving mode, XR contents may be reproduced on a display corresponding to interior windows, or front and rear windows of the vehicle.

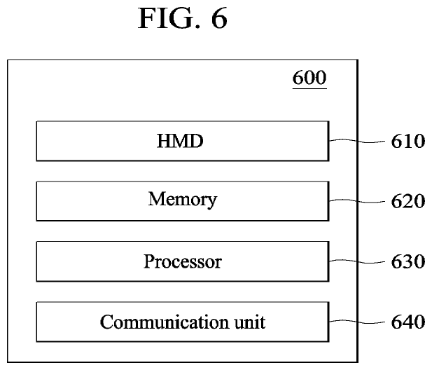

FIG. 6 is a block diagram of an XR apparatus 600 including an HMD according to an embodiment of the present invention.

The XR apparatus 600 according to an embodiment may include an HMD 610, a memory 620, a processor 630, and a communication unit 640.

The HMD 610 may be defined as an apparatus that generates image contents using a display element such as a liquid crystal display (LCD) element, and enlarges the generated image contents to a large screen to provide image contents to a wearer. The HMD 610 may be a mounted-type the display device on the head of the wearer, but the HMD 610 is not limited thereto. The HMD 610 may be similar to binoculars, or may have a light and thin structure such as ordinary glasses.

The HMD 610 according to one embodiment may monitor a condition of the passenger and display XR contents based on the movement of the vehicle. For example, while the passenger is viewing XR contents through the HMD 610, the HMD 610 may monitor at least one of a degree of pupil contraction of the passenger, pupil mobility, and whether or not the passenger is breaking out in a cold sweat. The condition of the passenger monitored by the HMD 610 is not limited thereto.

The memory 620 may store XR contents and programs for processing and controlling by the processor 630 and may store data input to or output from the XR apparatus 600.

The communication unit 640 may include one or more components that allow the XR apparatus 600 to communicate with an external network, a vehicle, and other devices (not illustrated). In addition, since the communication unit 640 according to the embodiment may be connected to the processor 630, the communication unit 640 may transmit or receive a signal between the processor 630 and at least one of the external network and the vehicle.

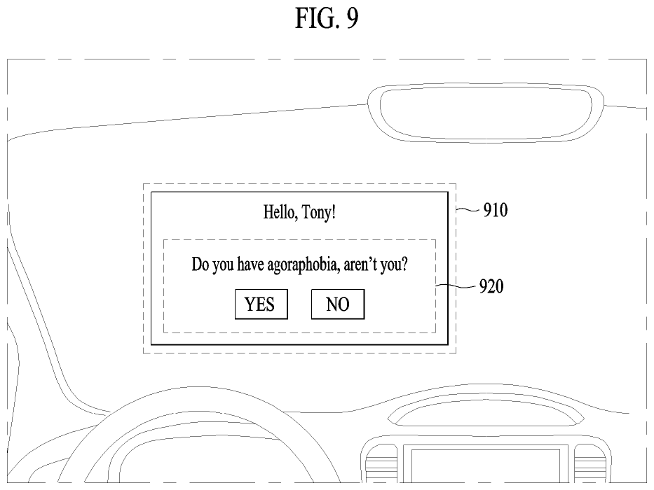

The processor 630 usually controls the overall operation of the XR apparatus 600. For example, the processor 630 may control the HMD 610, the communication unit 640, and the like in general by executing programs stored in the memory 620. In addition, as illustrated in FIGS. 9 to 16, the processor 630 may perform the functions of the XR apparatus 600 by executing programs stored in the memory 620.

In addition, the processor 630 according to an embodiment may check whether or not the passenger has a specific disease based on the health information of the passenger, determine whether or not the passenger has an abnormal symptom based on the check result and condition monitoring result of the passenger received from the HMD 610 and the vehicle, and make a request for XR contents based on at least one of the determination result and check result.

In addition, in a case where information about the predicted travel route of the vehicle and health information of the passenger are checked, the processor 630 according to another embodiment may determine whether a symptom-induced region as an area, in which the passenger is likely to have an abnormal symptom, is included among areas included in the predicted route, based on the health information of the passenger. Here, the health information of the passenger may be determined based on at least one of information received from the external network, information stored in the memory 620, and information input by the passenger.

In a case where it is determined that the predicted route includes a symptom-induced region, the processor 630 may perform at least one of making a request to the vehicle for another predicted route and outputting information about the symptom-induced region into the vehicle.

In addition, in the case where it is determined that the vehicle has reached the symptom-induced region or the passenger has an abnormal symptom, the processor 630 may output a message to guide the wearing of the HMD 610.

In addition, in the case where it is determined that the vehicle has reached the symptom-induced region or the passenger has an abnormal symptom, the processor 630 may output a control signal for increasing a period of monitoring the condition of the passenger, to the HMD 610 and a sensor installed in the vehicle, and in a case where it is determined that the vehicle gets out of the symptom-induced region or has overcome the abnormal symptom, the processor 630 may output a control signal for decreasing the period of monitoring the condition of the passenger thereto. In such an environment where the symptom is likely to be induced, it is possible to more rapidly respond to induction of the symptom by more frequently monitoring the condition of the passenger.

Meanwhile, the processor 630 may determine, as a new symptom-induced region, an area at the time when the passenger has the abnormal symptom on the travel route of the vehicle, and the processor 630 stores the new symptom-induced region in the memory 620.

In addition, the processor 630 may output a signal for controlling at least one of an air conditioning device, a directional spreading device, a lighting device, a sound device, and a seat controller of the passenger, based on the XR contents.

In addition, in the case where it is determined that the passenger has an abnormal symptom, the processor 630 may output at least one of a control signal on a travel speed of the vehicle or a signal indicating an emergency situation, when the abnormal symptom of the passenger persists even though a predetermined time has elapsed.

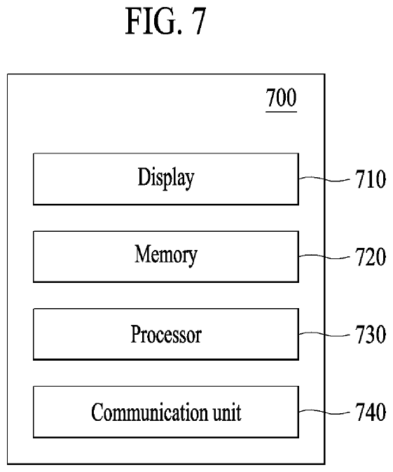

FIG. 7 is a block diagram of an XR apparatus 700 including a plurality of display units attached to a vehicle according to another embodiment of the present invention.

The XR apparatus 700 according to an embodiment may include a display 710, a memory 720, a processor 730, and a communication unit 740. The memory 720 and the communication unit 740 in FIG. 7 correspond to the memory 620 and the communication unit 640 in FIG. 6, respectively. Therefore, detailed description thereof will not be repeated.

The display 710 may include a plurality of display units attached to a windshield and window of the vehicle, and a component inside the vehicle. In addition, the display 710 may include a transparent display having predetermined transparency and displaying XR contents.

In order to have transparency, the transparent display may include at least one of a transparent Thin Film Electroluminescent (TFEL), a transparent Organic Light-Emitting Diode (OLED), a transparent Liquid Crystal Display (LCD), a transmissive transparent display, and a transparent Light Emitting Diode (LED), and the transparency of the transparent display may be adjusted. According to the embodiment, XR contents reproduced on the display located on the window of the vehicle may be provided with high transparency, in a case where the vehicle does not travel autonomously. Accordingly, the user may understand the surrounding environment together with XR contents. According to another embodiment, XR contents may not be reproduced on the display in the window of the vehicle in which the external information may be acquired in the case where the vehicle does not travel autonomously.

The processor 730 may check whether the passenger has a specific disease based on the health information of the passenger, determine whether or not the passenger has an abnormal symptom based on the check result and condition monitoring result of the passenger received from the vehicle, and make a request for XR contents based on at least one of the check result and determination result. In addition, the processor 730 may determine one or more display units included in the display 710 as a display unit for displaying XR contents, based on the location of the passenger.

In the case where it is determined that the predicted route includes the symptom-induced region, the processor 730 may perform at least one of making a request to the vehicle for another predicted route and outputting information about the symptom-induced region to the vehicle.

In addition, in the case where it is determined that the vehicle has reached the symptom-induced region or the passenger has an abnormal symptom while the vehicle is traveling, the processor 730 may output a message for inquiring about whether or not to reproduce XR contents using the display 710.

In addition, in the case where it is determined that the vehicle has reached the symptom-induced region or the passenger has an abnormal symptom, the processor 730 outputs a control signal for increasing a period of monitoring the condition of the passenger, and in the case where it is determined that the vehicle gets out of the symptom-induced region or the passenger has overcome the abnormal symptom, the processor 730 may output a control signal for decreasing the period of monitoring the condition of the passenger.

Meanwhile, the processor 730 may determine, as a new symptom-induced region, an area at the time when the passenger has the abnormal symptom on the travel route of the vehicle, and the processor 730 stores the new symptom-induced region in the memory 720.

In addition, the processor 730 may output a signal for controlling at least one of the air conditioning device, the directional spreading device, the lighting device, the sound device, and the seat controller of the passenger based on the XR contents.

In addition, in the case where it is determined that the passenger has an abnormal symptom, the processor 730 may output at least one of a control signal on the travel speed of the vehicle or a signal indicating an emergency situation, when the abnormal symptom of the passenger persists even though a predetermined time has elapsed.

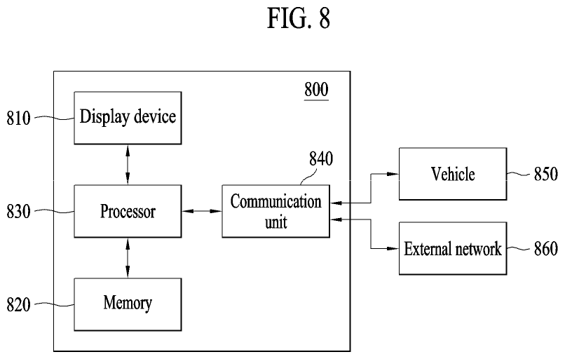

FIG. 8 is a diagram illustrating a signal flow between an XR apparatus 800 for a passenger and external components 850 and 860 according to still another embodiment of the present invention.

The XR apparatus 800 according to one embodiment may include a display device 810, a memory 820, a processor 830, and a communication unit 840. Here, the display device 810 may be defined as a term collectively referred to as the HMD 610 in FIG. 6 and the display portion 710 in FIG. 7.

The communication unit 840 of the XR apparatus 800 may be connected to the vehicle 850 and the external network 860 by wire or wireless, and may transmit or receive a signal therebetween.

For example, the communication unit 840 may transmit a vehicle control signal, a request for a predicted travel route, and the like to the vehicle 850, and may receive a condition monitoring result of the passenger, the predicted travel route, and the like from the vehicle 850. However, the information transmitted or received between the communication unit 840 and the vehicle 850 is not limited thereto.

Meanwhile, the communication unit 840 may transmit a request for the health information of the passenger, a request for XR contents, and the like to the external network 860, and receive the health information of the passenger and XR contents from the external network 860. However, the information transmitted or received between the external network 840 and the external network 860 is not limited thereto.

FIG. 9 is a view illustrating an example in which an XR apparatus checks whether or not a passenger is suffering from a specific disease based on the health information of the passenger according to an embodiment of the present invention.

The XR apparatus according to an embodiment of the present invention may receive the health information of the passenger on board the vehicle and may determine whether or not the passenger is suffering a specific disease based on the received information.

With reference to FIG. 9, the XR apparatus may identify a passenger on board the vehicle, and display the disease information of the passenger based on the health information stored in advance for the passenger. For example, in a case where the passenger on board the vehicle is “Tony”, and “Tony” of the passenger is checked as having agoraphobia by the checked of a previous boarding history, the XR apparatus may provide a message 910 for checking whether to have agoraphobia together with the passenger's name on the HUD of the vehicle once again.

In addition, in a case where the health information of the passenger on board the vehicle is not stored, the XR apparatus may receive the health information of the passenger through an external network, or prompt the passenger to enter the health information. In this case, an area 920 indicating the disease information of the passenger in FIG. 9 may be replaced with an area for inputting the symptom of the passenger. In the case where the health information of the passenger is received through the external network, the area 920 indicating the disease information of the passenger in FIG. 9 may be replaced with an area for querying the passenger whether to agree with reception of personal information.

Meanwhile, FIGS. 9 to 13 illustrate that a message is displayed on the HUD of the vehicle, but a displayed location of the message is not limited thereto. It is obvious to those skilled in the art that the message is not only displayed in one area of the vehicle and the XR apparatus, but also output in the form of a voice signal.

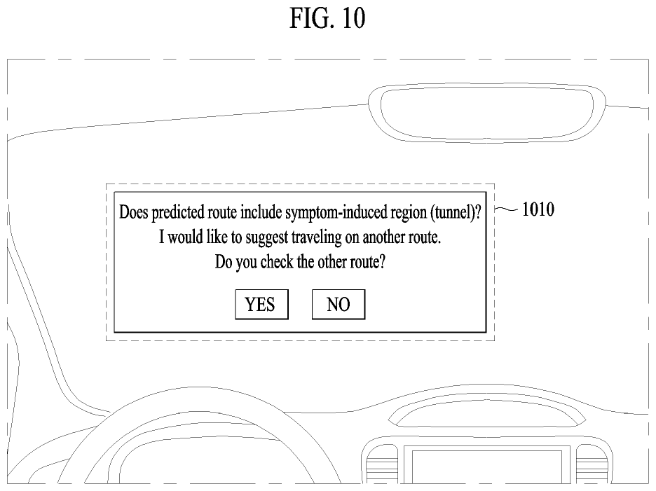

FIG. 10 is a view illustrating an example of a displayed message in a case where a predicted route of the vehicle includes a symptom-induced region according to an embodiment of the present invention.

The XR apparatus according to an embodiment of the present invention may determine whether or not the predicted route includes a symptom-induced region when the predicted travel route of the vehicle is received. Here, the symptom-induced region may be defined as an area in which it is determined that the passenger is likely to have an abnormal symptom based on the health information.

With reference to FIG. 10, the tunnel may be one of the symptom-induced regions for a passenger with diseases such as claustrophobia or agoraphobia. Therefore, in a case where the predicted travel route of the vehicle includes the tunnel, the XR apparatus notifies the passenger that the tunnel is included in the predicted route, and may suggest traveling on another travel route. In addition, the XR apparatus may make a request to the vehicle for the other travel route not including the symptom-induced region based on an input by the passenger.

As another example, the curved section may be one of the symptom-induced regions for a passenger with a severe motion sickness symptom. Therefore, in a case where the predicted travel route of the vehicle includes the curved section, the XR apparatus may notify the passenger that the predicted route includes the curved section, and may suggest traveling on another travel route or make a request to the vehicle for another travel route not including the symptom-induced region in response to an input by the passenger.

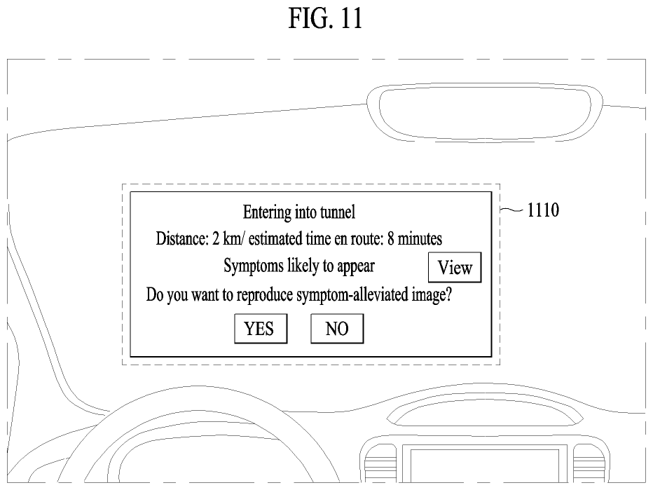

FIG. 11 is a view illustrating an example of a displayed message in a case where a vehicle reaches a symptom-induced region according to an embodiment of the present invention.

The XR apparatus according to an embodiment of the present invention may output information about the symptom-induced region to the passenger when the vehicle has reached the symptom-induced region while the vehicle is traveling.

With reference to FIG. 11, in the case where it is determined that the symptom-induced region is a tunnel based on the health information of the passenger, the XR apparatus may output information including the length information of the tunnel, an Estimated Time En route (ETE) required to get out of the tunnel, and the like before an entrance section into the tunnel. In addition, the XR apparatus may inquire of the passenger about whether or not to display the information about a symptom likely to appear when entering into the tunnel, whether or not to reproduce XR contents, and the like, based on the health information of the passenger.

Meanwhile, in FIG. 11, the message indicates that the information about the symptom-induced region, the inquiry about whether or not to display the information about an symptom likely to appear, and the inquiry about whether or not to reproduce XR contents are provided to the passenger at the same time. However, it is obvious to those skilled in the art that such information may be sequentially provided to the passenger.

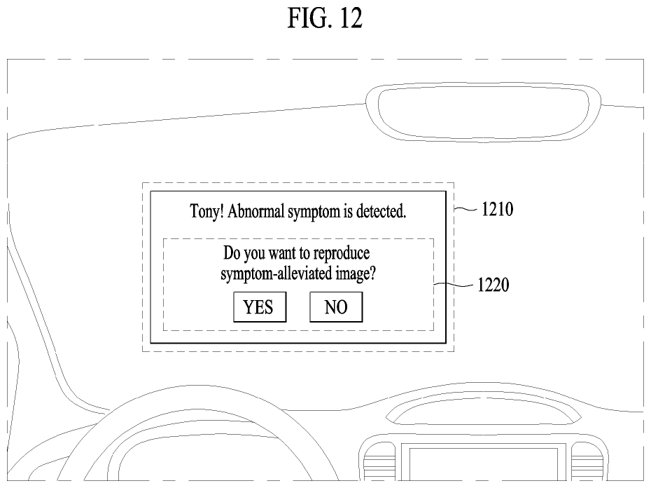

FIG. 12 is a view illustrating an example of a displayed message in a case where it is determined that the abnormal symptom of the passenger appears according to an embodiment of the present invention.

The XR apparatus according to an embodiment of the present invention may display XR contents, in a case where it is determined that the passenger has an abnormal symptom based on the condition monitoring result and health information of the passenger.

For example, with reference to FIG. 12, the XR apparatus may determine whether or not the passenger has an abnormal symptom based on the result of monitoring the condition of the passenger, and display the determination result. In addition, the XR apparatus may inquire of the passenger whether or not to reproduce XR contents selected based on the abnormal symptom of the passenger.

Here, after querying of the passenger about whether or not to reproduce XR contents, as illustrated in FIG. 12, the XR apparatus including the display may display XR contents based on an input by the passenger. In addition, first, the XR apparatus including the HMD may display the message to recommend the wearing of the HMD, and then display XR contents through the HMD.

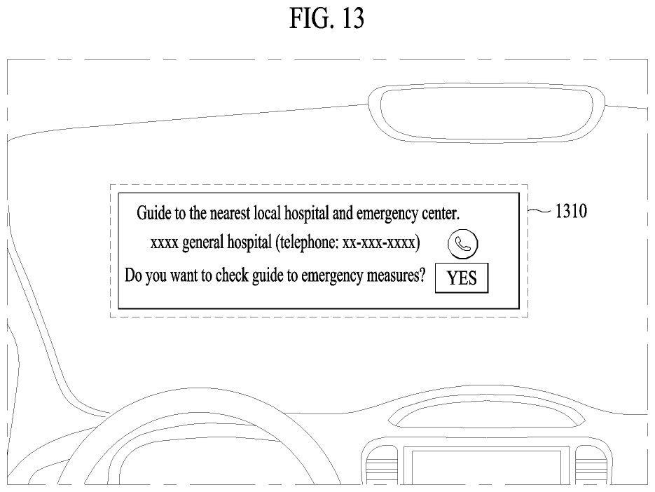

FIG. 13 is a view illustrating an example in which relevant information is provided such that a passenger is able to take emergency measures in a case where the abnormal symptom of the passenger is not improved, according to an embodiment of the present invention.

Even though the predetermined time has elapsed after it is determined that the passenger has an abnormal symptom, the XR apparatus according to an embodiment of the present invention may provide the passenger with information about emergency measures 1310, when it is determined that the symptom is not improved.

In the case where it is determined that the passenger has an abnormal symptom, the XR apparatus according to an embodiment may further take additional measures other than the measures of displaying XR contents. For example, in order to alleviate the abnormal symptom of the passenger, the XR apparatus may output a control signal to the air conditioning device and the directional spreading device of the vehicle to ventilate the inside of the vehicle or to spread fragrance. In addition, the XR apparatus may output a control signal to the lighting device of the vehicle, the sound device, and the seat controller of the passenger. In addition, the XR apparatus outputs the recommended speed information about the speed of the vehicle so as to improve abnormal symptom of the passenger, which allows a driver to adjust the speed of the vehicle or allows an autonomous driving vehicle to adjust the speed of the vehicle. One or more of the plurality of additional measures items may be entered from the passenger, or automatically selected by the XR apparatus in the case where the abnormal symptom of the passenger is not improved. In addition, one or more of the plurality of additional measures items may be provided to the passenger together when XR contents are reproduced.

When the abnormal symptom of the passenger is not improved even after these additional measures are taken, the XR apparatus may inquire of the passenger whether or not to display guide to the emergency measures for the abnormal symptom, as illustrated in FIG. 13. In addition, the XR apparatus may provide the passenger with information about the nearest hospital or Emergency Center based on the current location of the vehicle.

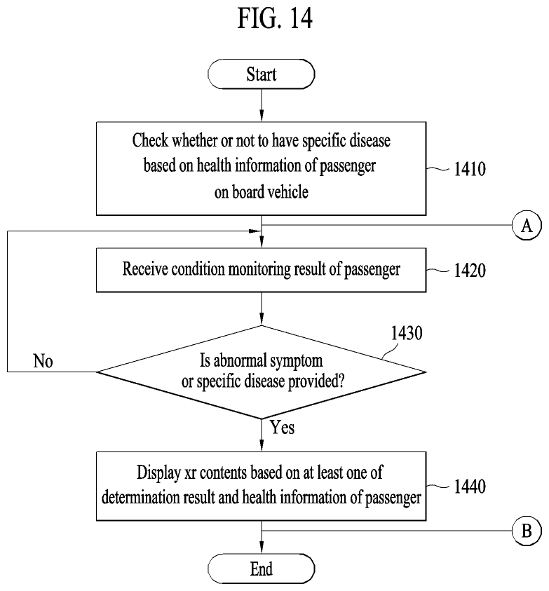

FIG. 14 is a flowchart illustrating a method of providing XR contents for a passenger in a vehicle, according to an embodiment of the present invention.

In step 1410, the XR apparatus may check whether or not the passenger has a specific disease based on the health information of the passenger on board the vehicle. At this time, the health information of the passenger may be stored in the XR apparatus in advance and may be received through the external network. Also, the health information of the passenger may be entered by the passenger. Specifically, based on the health information of the passenger, the XR apparatus may determine whether or not the passenger has a specific disease, whether or not the passenger has severe motion sickness, whether or not the passenger is a patient with dementia, whether or not the passenger is a child, and the like.

In step 1420, the XR apparatus may receive the condition monitoring result of the passenger. The XR apparatus may receive the condition monitoring result of the passenger from the sensor unit of the vehicle. The sensor unit of the vehicle may monitor the condition of the passenger including a gesture of the passenger, breathing capacity, heart rate, a body temperature, a facial expression and the like, and deliver the condition monitoring result to the XR apparatus. In a case where the XR apparatus includes the HMD, the XR apparatus may additionally monitor a condition of the passenger from the HMD, the condition including a degree of pupil contraction of the passenger, pupil mobility, and whether or not the passenger is breaking out in a cold sweat, and the like.

In step 1430, based on the received condition monitoring result of the passenger and the check result, the XR apparatus may determine whether or not the passenger has an abnormal symptom. For example, based on the specific disease information and the condition monitoring result of the passenger, the XR apparatus may determine whether or not an abnormal symptom of the passenger including an increase in heart rate or breathing capacity of the passenger, a forward bowing gesture of the passenger or a back bending gesture of the passenger, breaking out in a cold sweat or looking pale, putting a bag around his/her mouth, and the like has appeared. In the case where it is determined that the abnormal symptom of the passenger appears, the XR apparatus performs step 1440, and otherwise, the XR apparatus performs step 1420.

In step 1440, the XR apparatus may display XR contents based on at least one of the determination result and the health information of the passenger. For example, the XR apparatus may display XR contents in the case where the passenger has an abnormal symptom, and may display XR contents in the case where it is determined through the health information of the passenger that the passenger has severe motion sickness even though the abnormal symptom has not appeared. In addition, the XR apparatus may display XR contents reflecting the movement of the vehicle. Meanwhile, XR contents may be contents downloaded from the external network, and may be contents stored in the memory of the XR apparatus.

Meanwhile, steps 1420 to 1440 in FIG. 14 may be repeatedly performed while the vehicle is traveling.

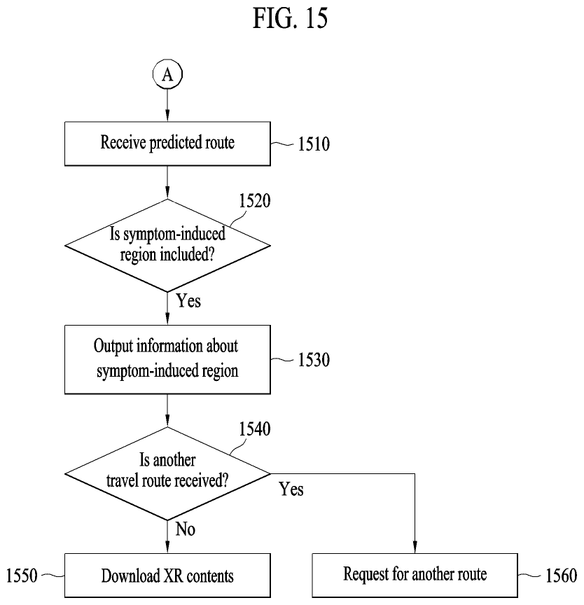

FIG. 15 is a flowchart illustrating a method performed by the XR apparatus to avoid a symptom of a passenger in a case where a predicted travel route of the vehicle is delivered, according to an embodiment of the present invention.

In step 1510, the XR apparatus may receive the predicted travel route of the vehicle from the vehicle.

In step 1520, the XR apparatus may determine whether a symptom-induced region as an area, in which the passenger is likely to have the abnormal symptom, is included among areas included in the received predicted route. In the case where it is determined that the predicted travel route includes the symptom-induced region, the XR apparatus performs step 1530, and otherwise, the method in FIG. 15 is ended, and the XR apparatus may proceed to step 1420 in FIG. 14.

In step 1530, the XR apparatus may output information about the symptom-induced region. Here, the information about the symptom-induced region may include a type of the symptom-induced region, a length thereof, and the Estimated Time En route (ETE) required to get out of the symptom-induced region. In addition, the XR apparatus may additionally output a message to recommend traveling on another travel route not including the symptom-induced region.

In step 1540, the XR apparatus may determine whether or not a request signal for another travel route not including the symptom-induced region is received from the passenger. In the case where the request signal of the other travel route is received from the passenger, the XR apparatus may perform step 1550, and otherwise, the XR apparatus may perform step 1560.

In step 1560, the XR apparatus makes a request to the vehicle for another travel route, and may perform step 1420 of FIG. 14.

In step 1550, the XR apparatus downloads XR contents that may alleviate a predicted symptom based on the health information of the passenger, and may perform step 1420 of FIG. 14. In a case where the XR apparatus performs step 1420 and its subsequent steps after performing step 1560, the XR apparatus may perform step 1440 when the abnormal symptom of the passenger not only appears but also the vehicle enters into the symptom-induced region in step 1430.

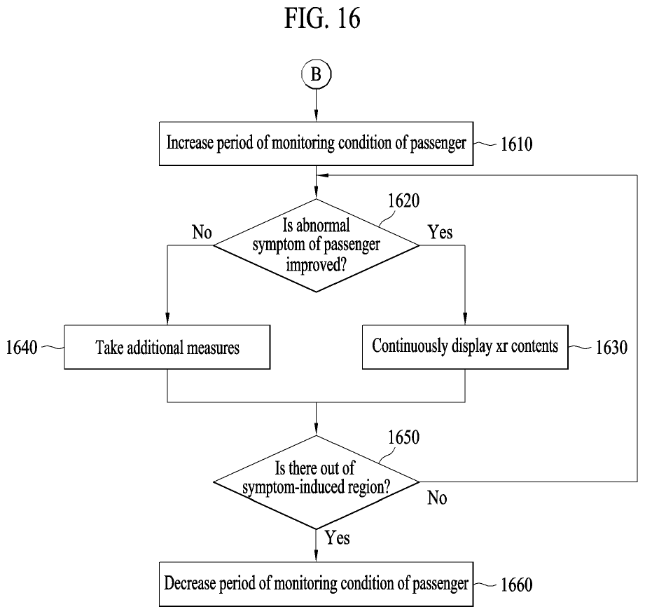

FIG. 16 is a flowchart illustrating a method performed by the XR apparatus to alleviate an abnormal symptom of a passenger, according to another disclosed embodiment.

In the case where it is determined that the abnormal symptom of the passenger appears or the vehicle enters into the symptom-induced region, the XR apparatus according to one embodiment may perform a method for improving the abnormal symptom of the passenger.

In step 1610, the XR apparatus may output a control signal for increasing a period of monitoring the condition of the passenger.

In step 1620, the XR apparatus may determine whether or not the abnormal symptom of the passenger is improved. In the case where it is determined that the abnormal symptom of the passenger is improved, step 1630 may be performed, and otherwise, step 1640 may be performed.

In step 1630, the XR apparatus may continuously display XR contents. In addition, the XR apparatus may continuously output the control signal for the vehicle that has been output for alleviating the symptom of the passenger.

In a case where the abnormal symptom of the passenger is not improved, in step 1640, the XR apparatus may further take additional measures, in addition to the currently displayed XR contents and the control signal for the vehicle that has been output for alleviating the symptom of the passenger. For example, in order to alleviate the abnormal symptom of the passenger, the XR apparatus may output a control signal to the air conditioning device and the directional spreading device of the vehicle to ventilate the inside of the vehicle or to spread fragrance. In addition, the XR apparatus may output a control signal to the lighting device of the vehicle, the sound device, and the seat controller of the passenger. In addition, the XR apparatus outputs the recommended speed information about the speed of the vehicle so as to improve abnormal symptom of the passenger, which allows a driver to adjust the speed of the vehicle or allows an autonomous driving vehicle to adjust the speed of the vehicle.

In step 1650, the XR apparatus may determine whether or not the vehicle gets out of the symptom-induced region. In a case where the vehicle gets out of the symptom-induced region, step 1660 may be performed, and otherwise, step 1620 may be performed.

In step 1660, the XR apparatus may output a control signal for decreasing the period of monitoring the condition of the passenger.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. An XR apparatus for a passenger in a vehicle, the XR apparatus comprising:
a head mounted display (HMD) configured to monitor a condition of the passenger and to display eXtended Reality (XR) contents based on a movement of the vehicle;
a memory configured to store the XR contents;
a processor configured to check whether or not the passenger has a specific disease based on the health information of the passenger, to determine whether or not the passenger has an abnormal symptom based on the check result and condition monitoring result of the passenger received from at least one of the HMD and the vehicle, and to make a request for XR contents based on at least one of the determination result and the health information of the passenger; and
a communication unit connected to the processor, and configured to transmit or receive a signal between the processor and at least one of an external network and the vehicle.

2. The XR apparatus of claim 1, wherein, in a case where information about a predicted travel route of the vehicle and health information of the passenger are received,
the processor determines whether a symptom-induced region as an area, in which the passenger is likely to have an abnormal symptom, is included among areas included in the predicted route, based on the health information of the passenger.

3. The XR apparatus of claim 2, wherein the health information of the passenger is received from the external network, stored in the memory, or input by the passenger.

4. The XR apparatus of claim 2, wherein, in a case where it is determined that the predicted route includes the symptom-induced region,
the processor performs at least one of making a request to the vehicle for another predicted route not including the symptom-induced region and outputting information about the symptom-induced region to the vehicle.

5. The XR apparatus of claim 2, wherein, in the case where it is determined that the vehicle has reached the symptom-induced region or the passenger has an abnormal symptom while the vehicle is traveling,
the processor outputs a message to guide the wearing of the HMD.

6. The XR apparatus of claim 2, wherein, in a case where it is determined that the vehicle has reached the symptom-induced region or the passenger has an abnormal symptom,
the processor outputs a control signal for increasing a period of monitoring a condition of the passenger, and
in a case where it is determined that the vehicle gets out of the symptom-induced region or the passenger has overcome the abnormal symptom,
the processor outputs a control signal for decreasing the period of monitoring the condition of the passenger.

7. The XR apparatus of claim 1, wherein the processor determines, as a new symptom-induced region, an area at the time when the passenger has the abnormal symptom on the travel route of the vehicle, and
the processor stores the new symptom-induced region in the memory.

8. The XR apparatus of claim 1, wherein the processor outputs a signal for controlling at least one of an air conditioning device, a directional spreading device, a lighting device, and a sound device of the vehicle, and a seat controller of the passenger, based on the XR contents.

9. The XR apparatus of claim 1, wherein, in the case where it is determined that the passenger has an abnormal symptom,
the processor outputs at least one of a control signal on the travel speed of the vehicle or a signal indicating an emergency situation, when the abnormal symptom of the passenger persists even though a predetermined time has elapsed.

10. An XR apparatus for a passenger in a vehicle, the XR apparatus comprising:
a display including a plurality of display units attached to a vehicle, and configured to display eXtended Reality (XR) contents based on a movement of the vehicle;
a memory configured to store the XR contents;
a processor configured to check whether or not the passenger has a specific disease based on the health information of the passenger, to determine whether or not the passenger has an abnormal symptom based on the checked result and condition monitoring result of the passenger received from the vehicle, to make a request for XR contents based on at least one of the determination result and the health information of the passenger, and to determine one or more display units included in the display as a display unit displaying the XR contents; and
a communication unit connected to the processor, and configured to transmit or receive a signal between the processor and at least one of an external network and the vehicle.

11. The XR apparatus of claim 10, wherein, in a case where information about a predicted travel route of the vehicle and health information of the passenger are received,
the processor determines whether a symptom-induced region as an area, in which the passenger is likely to have an abnormal symptom, is included among areas included in the predicted route, based on the health information of the passenger.

12. The XR apparatus of claim 11, wherein the health information of the passenger is received from the external network, stored in the memory, or input by the passenger.

13. The XR apparatus of claim 11, wherein, in a case where it is determined that the predicted route includes the symptom-induced region,
the processor performs at least one of making a request to the vehicle for another predicted route not including the symptom-induced region and outputting information about the symptom-induced region to the vehicle.

14. The XR apparatus of claim 11, wherein, in the case where it is determined that the vehicle has reached the symptom-induced region or the passenger has an abnormal symptom while the vehicle is traveling,
the processor outputs a message for inquiring about whether or not to reproduce the XR contents using the display.

15. The XR apparatus of claim 11, wherein, in a case where it is determined that the vehicle has reached the symptom-induced region or the passenger has an abnormal symptom,
the processor outputs a control signal for increasing a period of monitoring a condition of the passenger, and
in a case where it is determined that the vehicle gets out of the symptom-induced region or the passenger has overcome the abnormal symptom,
the processor outputs a control signal for decreasing the period of monitoring the condition of the passenger.

16. The XR apparatus of claim 10, wherein the processor determines, as a new symptom-induced region, an area at the time when the passenger has the abnormal symptom on the travel route of the vehicle, and
the processor stores the new symptom-induced region in the memory.

17. The XR apparatus of claim 10, wherein the processor outputs a signal for controlling at least one of an air conditioning device, a directional spreading device, a lighting device, and a sound device of the vehicle, and a seat controller of the passenger, based on the XR contents.

18. The XR apparatus of claim 10, wherein, in the case where it is determined that the passenger has an abnormal symptom, the processor outputs at least one of a control signal on the travel speed of the vehicle or a signal indicating an emergency situation, when the abnormal symptom of the passenger persists even though a predetermined time has elapsed.

19. A method of providing XR contents for a passenger in a vehicle, the method comprising:
checking whether or not the passenger has a specific disease based on health information of the passenger on board the vehicle;
receiving a condition monitoring result of the passenger from the vehicle;
determining whether or not the passenger has an abnormal symptom based on the received condition monitoring result of the passenger and the check result; and
displaying XR contents based on at least one of the determination result and the health information of the passenger.

20. The method of claim 19, further comprising:
receiving information about a predicted travel route of the vehicle;
determining whether a symptom-induced region as an area, in which the passenger is likely to have an abnormal symptom, is included among areas included in the received predicted route; and
outputting information about the symptom-induced region.
